(19) United States
(12) Patent Application Publication
(10) Pub. No.: US 2004/0213747 A1
(43) Pub. Date: Oct. 28, 2004

(54) Cosmetic compositions with film forming polymer

(76) Inventors: Anjali Abhimanyu Patil, Westfield, NJ (US); Joseph Frank Calello, Bridgewater, NJ (US)

Correspondence Address: Julie Blackburn, Revlon Consumer Products Corporation, 625 Madison Avenue, New York, NY, 10022, US

(21) Appl. No.: 10/423,624

(22) Filed: Apr. 26, 2003

(51) Int. Cl. A61K 7/021

(52) U.S. Cl. 424/63

(57) ABSTRACT

A cosmetic composition for coloring, conditioning, or beautifying keratinous surfaces comprising at least one film forming polymer which comprises vinyl ester monomers.

### TECHNICAL FIELD

The invention is in the field of cosmetic compositions for application to keratinous surfaces for the purpose of coloring, conditioning, or beautifying the keratinous surface.

### BACKGROUND OF THE INVENTION

Cosmetic formulators are constantly trying to improve wear, adhesion, and aesthetic properties of cosmetic compositions. For example, in color cosmetic compositions such as lipsticks, consumers are interested in formulas that wear well, feel comfortable on skin or lips, and provide nice visual aesthetics. Many consumers want a color cosmetic finish to exhibit a certain non-matte appearance because it provides lips that appear lush, dewy, and youthful. However, achieving long wearing properties often involves a trade off where comfort and shine must be sacrificed to achieve the desired long wearing properties. Cosmetic formulators have still not achieved the gold standard in long wearing lipsticks: a formula that provides long wear, has excellent gloss, and gives the same comfortable feel on the lips as a lip balm.

Long wearing features are desired in all types of color cosmetics including foundation makeup, eyeshadow, blush, eyeliner, lipstick, mascara, concealer, and so on. In the case of products which are not applied to the lips, the desire is to achieve long wear, while not impacting aesthetics such as finish, comfort, and removability.

Most unexpectedly, it has been discovered that a certain type of polymer, specifically a polymer having vinyl ester groups provides color cosmetic compositions that exhibit considerably better wear. In the case where the compositions are lipsticks, the use of the polymer comprised of vinyl alkyl ester monomers provides a very glossy finish, the polymer itself is compatible with the other ingredients traditionally used in such formulas, the composition is transfer resistant or long wearing depending on the other formula ingredients, and most importantly it is very comfortable on the keratinous surface to which it is applied.

It is an object of the invention to provide cosmetic compositions that have improved long wearing or transfer resistant properties, are comfortable to wear, and exhibit consumer desired visual aesthetics.

It is another object of the invention to provide a lipstick composition that has long wear, comfort, and a glossy finish. Preferably the long wearing lipstick is transfer resistant.

It is another object of the invention to provide a color cosmetic composition that provides long wear, a sheer, non-matte finish, and feels comfortable on the skin.

### SUMMARY OF THE INVENTION

The invention comprises a color cosmetic composition for application to keratinous surfaces comprising a film forming effective amount of a polymer comprised of vinyl ester monomers in an amount sufficient, by weight of the total polymer, to improve the glossiness of the color finish obtained when the cosmetic composition is applied to the keratinous surface.

The invention further comprises a cosmetic composition comprising a non-matting thixotropic agent in combination with a film forming effective amount of a polymer obtained by polymerizing ethylenically unsaturated monomers wherein at least some of the ethylenically unsaturated monomers are vinyl ester monomers.

The invention further comprises a cosmetic composition comprising a film forming effective amount of a polymer obtained by polymerizing ethylenically unsaturated monomers wherein at least some of the ethylenically unsaturated monomers are vinyl ester monomers and at least one plasticizer for the polymer.

The invention further comprises a cosmetic composition comprising a film forming effective amount of a polymer obtained by polymerizing ethylenically unsaturated monomers wherein at least some of the ethylenically unsaturated monomers are vinyl ester monomers, in combination with at least one polymer compatible liquid oil.

The invention further comprises a color cosmetic compositions comprising a film forming effective amount of a polymer obtained by polymerizing ethylenically unsaturated monomers wherein at least some of the ethylenically unsaturated monomers are vinyl ester monomers, in combination with at least one lipophilic particulate.

The invention further comprises a color cosmetic composition comprising a film forming effective amount of a polymer obtained by polymerizing ethylenically unsaturated monomers wherein at least some of the ethylenically unsaturated monomers are vinyl ester monomers, in combination with at least one hydrophilic particulate.

### DETAILED DESCRIPTION

In the percentages mentioned herein all percentages are by weight unless otherwise indicated. The term “cosmetic stick” means a self-supporting stick used for application of cosmetic films to the keratinous surface. The term “keratinous surface” means skin (including lips or eyelids), hair, or nails. The term “lipstick” means a composition for application to the lips to color, condition, or aesthetically improve the condition or appearance of the lips. The lipstick may be in the liquid, solid self-supporting form, or in the semi-solid paste or cream form. The composition of the invention may be in the anhydrous or emulsion form. If in the emulsion form the composition preferably comprises from about 0.01-95%, preferably 0.1-85%, more preferably 0.5-75% water and 0.01-95%, preferably about 0.1-85%, more preferably about 0.5-75% by weight of the total composition of an oily phase.

I. The Film Forming Polymer

The compositions of the invention comprise a film forming polymer in an amount effective to form a film on the keratinous surface to which the composition is applied. Preferably, the film forming polymer comprises at least some vinyl ester monomers, which are present in the polymer in an amount sufficient to impact the desired aesthetics of transfer resistance, long wear, and preferably, glossy finish. The film forming polymer is obtained by polymerizing ethylenically unsaturated monomers which comprise vinyl ester groups either alone or in combination with other monomers including silicon monomers, other ethylenically unsaturated monomers, or organic groups such as amides, urethanes, glycols, and the like. The various types of monomers or moieties may be incorporated into the film forming polymer by way of free radical polymerization, addition polymerization, or by formation of grafts and blocks which are attached to the growing polymer chain according to processes known in the art. In the preferred embodiment of the invention, the compositions comprise from about 0.001-85%, preferably about 0.05-75%, more preferably about 0.1-70% by weight of the total composition of the film forming polymer. Preferably the film forming polymer is an organic synthetic polymer obtained by polymerizing ethylenically unsaturated monomers comprised of vinyl ester groups and optionally organic or silicon groups or other types of ethylenically unsaturated monomers.

Typically, the film forming polymer comprises vinyl ester monomers having the following general formula:

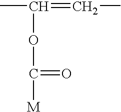

wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The film forming polymer may be a homopolymer or copolymer having the vinyl ester monomers either alone or in combination with other ethylenically unsaturated monomers, organic groups, or silicon monomers.

Suitable other monomers that may be copolymerized with the vinyl ester monomer include those having siloxane groups, including but not limited to those of the formula:

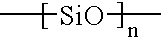

wherein n ranges from 1-1,000,000. The silicon monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

Also suitable are various types of organic groups that may be polymerized with the vinyl ester monomers including but not limited to urethane, amide, polyalkylene glycols, and the like.

One type of organic group that can be polymerized with the above monomers includes urethane monomers. Urethanes are generally formed by the reaction of polyhydroxy compounds with diisocyanates, as follows:

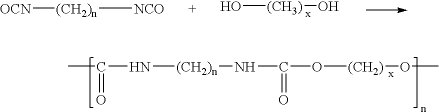

wherein n and x are each independently 1-10,000.

Another type of monomer that may be polymerized with the above comprise amide groups, preferably having the following general formula:

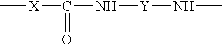

wherein X and Y are each independently linear or branched alkylene having 1-40 carbon atoms, which may be substituted with one or more amide, hydrogen, alkyl, aryl, or halogen substituents.

Another type of organic monomer may be alpha or beta pinenes, or terpenes, abietic acid, and the like.

Examples of suitable alkylene glycols that may be copolymerized with the vinyl ester monomers includes ethylene glycol, propylene glycol, butylene glycols, and the like.

The vinyl ester monomers may also be copolymerized with other ethylenically unsaturated monomers that are not vinyl esters, including those of the formula:

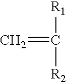

wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or —C(O)OM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

More preferred ethylenically unsaturated monomers are as above wherein R₁ is H or a C₁₋₃₀ alkyl, and R₂ is COOM wherein M is a C₁₋₃₀ straight or branched chain alkyl which may be substituted with one or more hydroxy groups.

More preferably, R₁ is H or CH₃, and R₂ is COOM wherein M is a C₁₋₁₀ straight or branched chain alkyl which may be substituted with one or more hydroxy groups.

Di-, tri- and polyfunctional monomers, as well as oligomers, of the above monofunctional monomers may also be copolymerized with the vinyl ester monomers. Suitable difunctional monomers include those having the general formula:

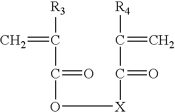

wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Suitable difunctional acrylates and methacrylates are those above where R₃ and R₄ are CH₃ and X is [(CH₂)ₓO_{y}]_{z} wherein x is 1-4; and y is 1-6; and z is 1-10.

Another type of difunctional acrylate or methacrylate is the compound above wherein R₃ and R₄ are CH₃ and X is [(CH₂)ₓO_{y}]_{z} wherein x is 2; and y is 1, and z is 4.

Trifunctional and polyfunctional monomers may be also be copolymerized with the vinyl ester monomers. Examples of such monomers include acrylates and methacrylates such as trimethylolpropane trimethacrylate or trimethylolpropane triacrylate.

Also suitable are cyclized monomers, in particular, cycloalkylacrylate monomers having the following general formulas:

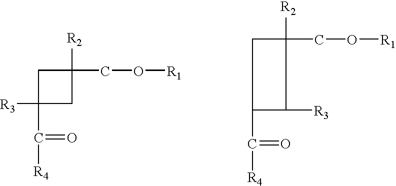

wherein R₁, R₂, R₃, and R₄ are as defined above.

The polymers used in the compositions of the invention can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference. Similarly, the polymers used in the compositions of the invention may also be made by preparing polymeric chains in accordance with the processes mentioned herein, then grafting the chains or blocks together according to methods well known in the art. Such polymers are often referred to as graft or block copolymers.

In the most preferred embodiment of the invention the film forming polymer is a homopolymer obtained by polymerizing ethylenically unsaturated monomers, all of which contain vinyl ester groups, or a copolymer comprised of vinyl ester monomers in combination with other types of ethylenically unsaturated monomers such as acrylic acid, methacrylic acid or their simple C1-22 esters. In the latter case, the polymer preferably comprises more than about 20% by weight of the polymerized vinyl ester monomers, more preferably from about 25 to 99%, even more preferably from about 50-99% of the vinyl ester monomers.

Examples of the most preferred polymers are homopolymers including poly vinyl fatty alkyl esters, polyvinyl short chain alkyl (C1-6) esters, and so on, including but not limited to those set forth herein:

Poly Vinyl Fatty Alkyl Esters

Suitable polyvinyl fatty alkyl esters include those where the alkyl chain is a fatty alkyl chain, generally having from 6 to 30 carbon atoms. In the case where the alkyl ester is a fatty alkyl, the fatty alkyl may be a saturated or unsaturated alkyl and the alkyl chain may be branched or straight. Examples of vinyl fatty ester radicals that are suitable include, but are not limited to, vinyl hexanoate, vinyl heptanoate, vinyl octanoate, vinyl nonanoate, vinyl isononanoate, vinyl decanoate, vinyl dodecanoate, vinyl laurate, vinyl stearate, vinyl isostearate, vinyl oleate, vinyl caprylate, vinyl behenate, vinyl palmitate, vinyl myristate, and so on.

One suitable film forming polymer includes polyvinyl neononanoate which may have a mixture of isomers, wherein the monomers are of the general formula:

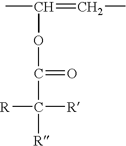

wherein R is H or an alkyl group having 7 carbon atoms, or R+R′+R″=an alkyl group having 7 carbon atoms. This polymer may be purchased from commercial sources including Sigma Aldrich, such polymer having Product No. 43,751-4 and the product name polyvinyl neononanoate, mixture of isomers. This polymer may also be purchased from Scientific Polymer Products, under product number 930 having product name poly(vinyl)neononanoate, which is described as a powder material having an approximate molecular weight of about 14,000, and being soluble in the solvents benzene, chloroform, cyclohexane, hexane, MEK (methyl ethyl ketone), methylene chloride, THF, toluene, and xylene.

Another example of a polyvinyl fatty alkyl ester that is suitable for use in the claimed compositions is polyvinyl laurate, a polymer sold by Scientific Polymer Products, Inc. under catalog number 302, having a CAS number 26246-97-3.

Another example of a suitable polymer includes poly(vinyl neodecanoate), which is also sold by Scientific Polymer Products under catalog number 267, and has the CAS number 66987-22-2.

Poly Vinyl Short Chain Alkyl Esters

Also suitable are polyvinyl short chain alkyl esters, generally having from one to five carbon atoms. Examples include vinyl ethanoate, vinyl propionate, vinyl butanoate, vinyl pentanoate, vinyl pivalate, and the like. One example of a polyvinyl short chain alkyl ester is polyvinyl propionate, available from Scientific Polymer Products, Inc., having CAS No. 25035-841.

Another example of a suitable polyvinyl short chain alkyl ester is polyvinyl pivalate, which comprises monomers having the general formula:

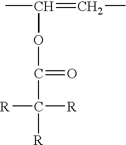

wherein R is methyl.

The preferred film forming polymers used in the compositions of the invention may be in the liquid, solid, or semi-solid form. Preferably the polymers are in the solid form and may be dispersed or solvated in volatile or non-volatile oils or water.

In the most preferred embodiment of the invention, the film forming polymer is branched chain alkyl ester, specifically polyvinyl neononanoate.

II. Other Ingredients

The compositions of the invention may comprise other ingredients such as pigments, fillers, thixotropic agents, plasticizers, emulsifiers, humectants, vitamins, antioxidants and so forth. Examples of such other ingredients include:

A. Pigments and Particulate Fillers

The composition of the invention preferably comprises about 0.05-30%, preferably about 0.1-25%, more preferably about 0.5-20% by weight of the total composition of one or more pigments which may be inorganic, or organic pigments or salts of organic pigments. Particularly preferred organic pigments are red, green, blue, yellow, violet, orange, and mixtures thereof. Also suitable are Lakes of such pigments, which means that the organic pigments are reacted with a metal salt such as calcium, aluminum, barium, zirconium, and the like to form salts. Particularly preferred are aluminum Lakes of the organic pigments, which is where the organic pigment is reacted with aluminum to form the aluminum salt. Formation of the metal salt of the organic pigment will generally also convert the pigment from a water soluble pigment into a water insoluble pigment, and such pigments tend to become lipophilic in nature, meaning that they will exhibit affinity for lipophilic or oily ingredients in the composition. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors which are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

Also suitable are inorganic pigments that include iron oxides such as red, blue, black, green, and yellow; titanium dioxide, bismuth oxychloride, and the like. Preferred are iron oxides.

It may also be desirable to include one or more particulate fillers in the claimed composition. If so, suggested ranges are about 0.001-40%, preferably about 0.05-35%, more preferably about 0.1-30% by weight of the total composition. Preferably, the particulate fillers include non-pigmentitious particles that generally have a particle size ranging from about 0.02 to 200, preferably 0.5 to 100, microns. Suitable particle fillers include titanated mica, fumed silica, spherical silica, polymethylmethacrylate, micronized teflon, boron nitride, mica, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The particulates may also be in the fiber form, such as cellulose fibers, rayon fibers, nylon or silk fibers and the like. Such fibers are generally circular in cross-section and have a discernable length. Preferably the length ranges from 1 to 5 mm.

The above mentioned pigments, powders or fibers may be inherently lipophilic or hydrophilic. The term “lipophilic” when used in this context means that the particulates will exhibit an affinity for lipophilic ingredients, or will be soluble or dispersible in lipophilic ingredients such as nonpolar oils. The term “hydrophilic” when used in this context means that the particulates exhibit an affinity for, or are soluble or dispersible in, water. For example, many types of organic pigments are hydrophilic and will be soluble or dispersible in water. On the other hand, in some cases, if the organic pigments are reacted with metal salts to form Lakes, in some cases the pigments will then exhibit a more lipophilic character. In the compositions of the invention, the pigments may be inherently lipophilic or inherently hydrophilic, depending on the properties desired. Further, if the pigments, particles or fibers are hydrophilic or if it is desired to increase their already lipophilic character, it may be desired to further surface treat the particulates with lecithin, amino acids, mineral oil, silicone oil or various other agents either alone or in combination, which coat the particulate surface and render the particles more lipophilic in nature. The term “lipophilic” means that the pigment or particles will be compatible with the lipophilic or oily phase of the composition. In the case of an emulsion, a lipophilic particle will have an affinity for the oily phase of the emulsion.

In the most preferred embodiment of the invention the composition comprises a mixture of organic and inorganic pigments in an amount sufficient to provide color to the composition. Such pigments may be hydrophilic or lipophilic in character.

B. Liquid Carrier

The composition preferably comprises about 0.1-85%, preferably about 5-80%, more preferably about 10-75% by weight of the total composition of a liquid carrier for the film forming polymer and the pigments, which may comprise one or more oils or other liquid materials including water. Preferably the liquid carrier is anhydrous. The term “anhydrous” means that water is not intentionally added to the composition. A variety of ingredients may be suitable including volatile oils, nonvolatile oils, and mixtures thereof. Examples of suitable liquid carriers include:

1. Volatile Liquids

The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C. Preferably, the compositions of the invention contain a significant portion of volatile solvents as the liquid carrier. When the composition of the invention is applied to the desired surface, the volatile solvent of the invention must be capable of flashing off to leave the other ingredients in the composition affixed to the surface. Suitable volatile solvents generally have a viscosity ranging from about 0.5 to 10 centipoise at 25° C. Suitable volatile solvents include linear silicones, cyclic silicones, paraffinic hydrocarbons, or mixtures thereof.

(a). Volatile Silicones

Cyclic silicones (or cyclomethicones) are of the general formula:

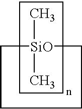

where n=3-6.

Linear volatile silicones in accordance with the invention have the general formula:

(CH₃)₃Si—O—[Si(CH₃)₂—O]ₙ—Si(CH₃)₃

where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids include octamethylcyclotetrasiloxane, octamethyltrisiloxane, decamethyltetrasiloxane, decamethylcyclopentasiloxane, hexamethyldisiloxane, and mixtures thereof.

(b). Paraffinic Hydrocarbons

Also suitable as the volatile liquid are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. Examples of suitable volatile paraffinic hydrocarbons include isohexadecane, isododecane, or mixtures thereof.

2. Nonvolatile Liquids

The liquid carrier may also comprise low viscosity non-volatile liquid oils such as silicones, esters, and the like. If the nonvolatile oils are too heavy or greasy it may hamper the desired long wearing characteristics of the invention. Generally, the viscosity of the nonvolatile oils if present should range from about 11-1000, preferably less than 100 centipoise, most preferably less than about 50 centipoise at 25° C. Examples of such oils include polyalkylsiloxanes, polyarylsiloxanes, and polyethersiloxanes. Examples of such nonvolatile silicones are disclosed in Cosmetics, Science and Technology 27-104 (Balsam and Sagarin ed. 1972); and U.S. Pat. Nos. 4,202,879 and 5,069,897, both of which are hereby incorporated by references. Further nonlimiting examples of such silicones include dimethicone, phenyl trimethicone, dimethicone copolyol, and so on.

Also suitable are lower viscosity organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety. Such silicones or organic oils include those further described as follows:

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, ie. may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(ii). Diesters

Suitable diesters that may be used in the compositions of the invention are the reaction product of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be subsituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(iii). Triesters

Suitable triesters comprise the reaction product of a tricarboxylic acid and an aliphatic or aromatic alcohol. As with the mono- and diesters mentioned above, the acid and alcohol contain 2 to 30 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, and so on.

(b). Hydrocarbon Oils.

It may be desirable to incorporate one or more non-volatile hydrocarbon oils into the composition of the invention. The term “nonvolatile” means that the oil has a vapor pressure of less than about 2 mm. of mercury at 20° C.

Suitable nonvolatile hydrocarbon oils include isoparaffins and olefins, preferably those having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, mineral oil, pentahydrosqualene, squalene, squalane, and mixtures thereof.

(c). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(d). Glyceryl Esters of Fatty Acids

The nonvolatile oil may also comprise naturally occuring glyceryl esters of fatty acids, or triglycerides. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(e). Nonvolatile Silicones

Nonvolatile silicone oils, both water soluble and water insoluble, are also suitable for use as the non-volatile oil. Such silicones preferably have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylhexamethicone, phenyl trimethicone, or polyphenylmethylsiloxane; dimethicone, alkyl substituted dimethicones, and mixtures thereof.

Water soluble, non-film forming silicones such as dimethicone copolyol, dimethiconol, and the like may be used. Such silicones are available from Dow Corning as the 3225C formulation aid, Dow 190 and 193 fluids, or similar products marketed by Goldschmidt under the ABIL tradename.

(f). Fluorinated Oils

Also suitable as the oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester which is formed by the reaction of a guerbet alcohol having the general formula:

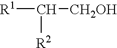

and a fluoroalcohol having the following general formula:

CF₃—(CF₂)ₙ—CH₂—CH₂—OH

wherein n is from 3 to 40.

with a carboxylic acid having the general formula:

R³COOH, or

HOOC—R³—COOH

wherein R₁, R², and R³ are each independently a straight or branched chain alkyl.

Preferably, the guerbet ester is a fluoro-guerbet ester which is formed by the reaction of a guerbet alcohol and carboxylic acid (as defined above), and a fluoroalcohol having the following general formula:

CF₃—(CF₂)ₙ—CH₂—CH₂—OH

wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. One type of such an ester is fluorooctyldodecyl meadowfoamate, sold under the tradename Silube GME-F by Siltech, Norcross Ga.

In the preferred composition the liquid carrier comprises one or more volatile liquids either alone or in combination with one or more nonvolatile liquids. Particularly preferred is where the liquid vehicle comprises volatile silicone or volatile paraffinic hydrocarbons which comprises the carrier in which the pigments and film forming polymer are dispersed.

C. Thixotropic Agents

Preferably the compositions of the invention comprise one or more thixotropic agents. The term “thixotropic agent” means an ingredient or combination of ingredients that increase the viscosity of, or thicken, the composition. In the most preferred embodiment of the invention, the thixotropic agent is a non-matting thixotropic agent, which means that it exhibits a reduced tendency to mute or matte the glossy effect achieved with the film forming polymer. Suggested ranges of thixotropic agent range from about 0.01-60%, preferably about 0.05-50%, more preferably about 0.1-45% by weight of the total composition.

One type of non-matting thixotropic agent comprises natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quaternium-18 hectorite.

Also suitable as thixotropic agents are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl Another type of viscosity modifier that may be used in the compositions are silicas, silicates, silica silylate, and derivatives thereof. These silicas and silicates are generally found in the particulate form. Particularly preferred is silica.

Also suitable thixotropic agents include cross-linked organosiloxane compounds also known as silicone elastomers. Such elastomers are generally prepared by reacting a dimethyl methylhydrogen siloxane with a crosslinking group comprised of a siloxane having an alkylene group having terminal olefinic unsaturation or with an organic group having an alpha or omega diene. Examples of suitable silicone elastomers for use as thixotropic agents include Dow Corning 9040, sold by Dow Corning, and various elastomeric silicones sold by Shin-Etsu under the KSG tradename including KSG 15, KSG 16, KSG 19 and so on.

Suitable thixotropic agents include natural or synthetic waxes. A variety of waxes are suitable including animal, vegetable, mineral, or silicone waxes. Generally such waxes have a melting point ranging from about 28 to 125° C., preferably about 30 to 100° C. Examples of waxes include acacia, beeswax, ceresin, cetyl esters, flower wax, citrus wax, camauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on. Suitable waxes are set forth in U.S. Pat. No. 5,725,845, which is hereby incorporated by reference in its entirety. Preferred ranges of wax are from about 0.01-75%, preferably about 1-65% by weight of the total composition.

E. Plasticizers

It may be desirable to incorporate one more plasticizers into the composition. Plasticizers may improve the spreadability and application of the composition to the surface to which it is applied and in some cases will interact with the film forming polymer to make it more flexible. If present, the plasticizer may be found in the oil or water phase if the composition of the invention is in the form of an emulsion, and in the oil or lipophilic phase if the composition is in the anhydrous form. Suggested ranges of plasticizers range from about 0.01-60%, preferably about 0.05-15%, more preferably about 0.1-10% by weight of the total composition. A variety of plasticizers are suitable including Suitable plasticizers include glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like. Also suitable, are plasticizers of the following general formula:

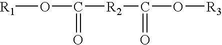

wherein R₁, R₂, and R₃ are each independently a C₁₋₂₀ straight or branched chain alkyl or alkylene which may be substituted with one or more hydroxyl groups. Preferably, R₁ is a C₃₋₁₀ straight or branched chain alkyl; R₂ is a C₂₋₈ alkyl which may be substituted with one or more hydroxyl groups; and R₃ is a C₃₋₁₀ straight or branched chain alkyl. Examples of such compounds include dioctyl malate, diisopropyl adipate, dibutyl adipate, dibutyl sebacate, dioactyl azelate, dioctyl succinate, dioctyl fumarate, and the like.

F. Other Film Forming Polymers

It may be desirable to include one or more ancillary film forming polymers in the compositions in addition to the polymer comprised of vinyl ester monomers. If so, a variety of polymers may be used including:

1. Polymers from Ethylenically Unsaturated Monomers

Also suitable for use as ancillary film forming polymers are polymers made by polymerizing one or more ethylenically unsaturated monomers that are not vinyl ester monomers, either alone or in combination with various types of organic groups, including but not limited to urethane, amides, alkylene glycols, etc. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on. Such polymers may be water soluble or dispersible, or oil soluble or dispersible in oil.

One type of suitable polymer includes those which contain monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers that may be used to form the other film forming polymer include those of the formula:

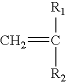

wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or C(O)OM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

Similarly, di-, tri- and polyfunctional monomers, as well as oligomers, of the above monofunctional monomers may also be used. Suitable difunctional monomers include those having the general formula:

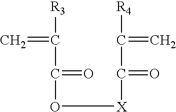

wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100.

Trifunctional and polyfunctional monomers are also suitable for use in the other film forming polymer. Examples of such monomers include acrylates and methacrylates such as trimethylolpropane trimethacrylate or trimethylolpropane triacrylate.

The other film forming polymers, if present in the compositions of the invention, can be prepared as noted above with respect to the film forming polymer containing the vinyl ester monomers.

Also suitable are polymers which are cyclized, in particular, cycloalkylacrylate polymers or copolymers having the following general formulas:

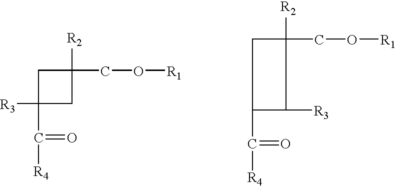

wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane.

Such monomers may be copolymerized with various types of organic groups including but not limited to urethane, amide, polyalkylene glycols, and the like as noted above with respect to the vinyl ester containing polymer.

2. Silicone Polymers

Also suitable are various types of water soluble or water insoluble (oil soluble) high molecular weight silicone polymers such as silicone gums, resins, and the like.

Suitable silicone resins include siloxy silicate polymers having the following general formula:

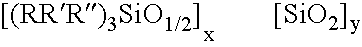

wherein R, R′ and R″ are each independently a C₁₋₁₀ straight or branched chain alkyl or phenyl, and x and y are such that the ratio of (RR′R″)₃SiO_{1/2} units to SiO₂ units is 0.5 to 1 to 1.5 to 1.

Preferably R, R′ and R″ are a C₁₋₆ alkyl, and more preferably are methyl and x and y are such that the ratio of (CH₃)₃SiO_{1/2} units to SiO₂ units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the tradename 2-0749 and 2-0747, which is a blend of about 40-60% volatile silicone and 40-60% trimethylsiloxy silicate. Dow Corning 2-0749 in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41. A similar siloxysilicate resin is available from GE Silicones under the tradename SR1000 and is a fine particulate solid material.

Another type of silicone resin is referred to as a T or MT resin, and has the general formula:

(R₁SiO_{3/2})ₓ

where x ranges from about 1 to 100,000, preferably about 1-50,000, more preferably about 1-10,000, and wherein R₁ is independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆); or hydrogen. Typically T or MT silicones are referred to as silsesquioxanes, and in the case where M units are present methylsilsesquioxanes. One type of such resin

is manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polysilsesquioxanes that may be used as the film forming polymer include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Another type of silicone resin suitable for use in the invention comprises the silicone esters set forth in U.S. Pat. No. 5,725,845 which is hereby incorporated by reference in its entirety. Other polymers that can enhance adhesion to skin include silicone esters comprising units of the general formula RₐR^{E}_{b}SiO_{[4-(a+b)/2]} or R¹³ₓR^{E}_{y}SiO_{1/2} wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieities (e.g. of about 2, often about 3 to 10 carbon atoms) and one or more aliphatic alcohol moieities (e.g. of about 10 to 30 carbon atoms). Examples of such acid moieities include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety.

Preferably the silicone ester will have a melting point of no higher than about 90° C. It can be a liquid or solid at room temperature. Preferably it will have a waxy feel and a molecular weight of no more than about 100,000 Daltons.

Silicone esters having the above formula are disclosed in U.S. Pat. No. 4,725,658 and U.S. Pat. No. 5,334,737, which are hereby incorporated by reference. Preferred silicone esters are the liquid siloxy silicates disclosed in U.S. Pat. No. 5,334,737, e.g. diisostearoyl trimethylolpropane siloxysilicate (prepared in Examples 9 and 14 of this patent), and dilauroyl trimethylolpropane siloxy silicate (prepared in Example 5 of the patent), which are commercially available from General Electric under the tradenames SF 1318 and SF 1312, respectively.

Silicone gums or other types of silicone solids may be used provided they are soluble in the liquid vehicle. Examples of silicone gums include those set forth in U.S. Pat. No. 6,139,823, which is hereby incorporated by reference. Preferred gums have a viscosity of 600,000 to 1,000,000 centipoise at 25° C.

3. Natural Polymers

Also suitable for use are one or more naturally occurring water soluble or oil soluble polymeric materials such as resinous plant extracts including such as rosin, shellac, and the like.

The invention will be described in connection with the following examples, which are set forth for the purposes of illustration only.

### EXAMPLE 1

A transfer resistant, shiny liquid lipstick was prepared as follows:

| | % by weight |
|---|---|
| Polyvinyl neononanoate | 35.7 |
| Dimethicone (1 centistoke) | 51.45 |
| Methoxypropanol | 3.60 |
| Pigment/Pearls/Mica | 8.75 |
| Quaternium-18 Hectorite | 0.50 |

The composition was prepared by grinding the pigments in a portion of the volatile oils, then combining the ground pigments with the remaining ingredients in the composition to form a semi-solid lip color.

### EXAMPLE 2

A liquid composition suitable for use as eyeliner was made as follows:

| | % by weight |
|---|---|
| Isododecane | 26.45 |
| Polyvinyl neononanoate | 25.0 |
| Polyehtylene | 5.0 |
| Blue 1 lake | 4.00 |
| Red 40 lake | 3.60 |
| Yellow 5 lake | 0.80 |
| Green 5 | 0.05 |
| Silica | 7.00 |
| Isododecane/quaternium-18 hectorite | 25.80 |
| propylene carbonate | |
| Acetyl tributyl citrate | 1.6 |
| Methylparaben | 0.35 |
| Dehydroacetic acid | 0.20 |
| Propyl paraben | 0.10 |
| Sorbic acid | 0.05 |

### EXAMPLE 3

A clear nail enamel was prepared as follows:

| | % by weight |
|---|---|
| Ethyl acetate | 44.4 |
| Butyl acetate | 8.9 |
| Polyvinyl neononanoate | 44.4 |
| Acetyl tributyl citrate | 2.3 |

The composition was prepared by combining the ingredients and mixing well.

### EXAMPLE 4

A shiny lip gel was prepared as follows:

| | % by weight |
|---|---|
| Polyvinyl neononanoate | 30.0 |
| Isododecane | 44.0 |
| Isododecane/quaternium-18 hectorite | 15.0 |
| propylene carbonate | |
| Polyethylene | 1.0 |
| Pigments/Pearls/Mica | 10.0 |

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A color cosmetic composition for application to keratinous surfaces comprising a film forming effective amount of a polymer comprised of vinyl ester monomers in an amount sufficient, by weight of the total polymer, to improve the glossiness of the color finish obtained when the cosmetic composition is applied to the keratinous surface.

2. The composition of claim 1 wherein at least some of the vinyl ester monomers are fatty esters.

3. The composition of claim 2 wherein the fatty esters are straight or branched chain fatty esters.

4. The composition of claim 2 wherein the fatty esters have at least some unsaturation in the alkyl chain.

5. The composition of claim 1 wherein the polymer comprises at least about twenty percent by weight of the total polymer of vinyl ester monomers.

6. The composition of claim 2 wherein the vinyl ester monomer comprises vinyl fatty alkyl ester monomers wherein the fatty chain has from about 6 to 30 carbon atoms.

7. The composition of claim 6 wherein the fatty ester group in the vinyl ester monomers is selected from the group consisting of hexanoate, heptanoate, octanoate, nonanoate, isononanoate, decanoate, dodecanoate, palmitate, myristate, oleate, caprylate, stearate, isostearate, laurate, behenate, and mixtures thereof.

8. The composition of claim 1 wherein the film forming polymer is a homopolymer of vinyl fatty alkyl ester monomers.

9. The composition of claim 1 wherein the film forming polymer is a copolymer of vinyl fatty alkyl ester monomers and one or more monomers selected from the group consisting of ethylenically unsaturated monomers that are not vinyl esters, silicon monomers, organic groups, and mixtures thereof.

10. The composition of claim 9 wherein the film forming copolymer is a copolymer of vinyl fatty alkyl ester monomers and one or more ethyenically unsaturated monomers that are not vinyl ester monomers.

11. The composition of claim 10 wherein the other ethylenically unsaturated monomers are acrylic acid, methacrylic acid, or their simple esters.

12. The composition of claim 1 wherein the vinyl ester monomers are C₁₋₅ alkyl ester monomers.

13. The composition of claim 12 wherein the vinyl ester monomer comprises vinyl propionate and the polymer is polyvinyl propionate.

14. The composition of claim 12 wherein the monomer comprises vinyl pivilate and the polymer is polyvinyl pivilate.

15. The composition of claim 1 wherein the monomer comprises vinyl neononanoate and the polymer is polyvinylneononanoate.

16. The composition of claim 1 wherein the monomer comprises vinyl neodecanoate and the polymer is polyvinylneodecanoate.

17. The composition of claim 1 wherein the vinyl ester monomer comprises an ethylenically unsaturated monomer having an OCOM group attached to one of the carbon atoms in the ethylenically unsaturated group, wherein 0 is oxygen, C is carbon, and M is a substituted or unsubstituted straight or branched chain C₁₋₃₀ alkyl wherein the substituents are halogen, hydroxyl, alkylcarbonyloxy, alkyloxycarbonyl,

18. A cosmetic composition comprising a non-matting thixotropic agent in combination with a film forming effective amount of a polymer obtained by polymerizing ethylenically unsaturated monomers wherein at least some of the ethylenically unsaturated monomers are vinyl ester monomers.

18. The composition of claim 17 wherein the film forming polymer comprises greater than about twenty percent by weight of the total polymer of vinyl ester monomers.

19. The composition of claim 17 wherein the polymer is a homopolymer of vinyl ester monomers.

20. The composition of claim 17 wherein the polymer is a copolymer of vinyl ester monomers and silicon monomers, ethylenically unsaturated monomers that do not have vinyl ester groups, or organic groups.

21. The composition of claim 19 wherein the homopolymer comprises vinyl ester monomers where the ester groups are fatty ester groups.

22. The composition of claim 21 wherein the fatty ester groups are selected from the group consisting of hexanoate, heptanoate, octanoate, nonanoate, isononanoate, decanoate, dodecanoate, palmitate, myristate, oleate, caprylate, stearate, isostearate, laurate, behenate, and mixtures thereof.

23. The composition of claim 17 wherein the vinyl ester groups are C₁₋₅ alkyl esters.

24. The composition of claim 23 wherein the vinyl ester groups are propionate, pivalate, butanoate, heptanoate, acetate, or mixtures thereof.

25. The composition of claim 17 wherein the non-matting thixotropic agent comprises natural or synthetic montmorillonite minerals.

26. The composition of claim 25 wherein the natural or synthetic montmorillonite mineral is quaternized.

27. The composition of claim 26 wherein the synthetic montmorillonite mineral comprises a quaternized hectorite, bentonite, or mixtures thereof.

28. The composition of claim 17 wherein the non-matting thixotropic agent is Quaternium-18 hectorite.

29. A cosmetic composition comprising a film forming effective amount of a polymer obtained by polymerizing ethylenically unsaturated monomers wherein at least some of the ethylenically unsaturated monomers are vinyl ester monomers and at least one plasticizer for the polymer.

30. The composition of claim 29 wherein the plasticizer is a glyceryl, glycol, or citrate ester.

31. The composition of claim 29 wherein the plasticizer is a compound of the formula: 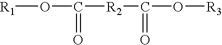
wherein R₁, R₂, and R₃ are each independently a C₁₋₂₀ straight or branched chain alkyl or alkylene which may be substituted with one or more hydroxyl groups.

32. A cosmetic composition comprising a film forming effective amount of a polymer obtained by polymerizing ethylenically unsaturated monomers wherein at least some of the ethylenically unsaturated monomers are vinyl ester monomers, in combination with at least polymer compatible liquid oil.

33. The composition of claim 32 wherein the polymer compatible liquid oil comprises a volatile silicone, a volatile paraffinic hydrocarbon, or mixtures thereof.

34. The composition of claim 32 wherein the polymer compatible liquid oil comprises a non-volatile silicone oil.

35. The composition of claim 32 wherein the polymer compatible liquid oil comprises an organic oil.

36. A color cosmetic compositions comprising a film forming effective amount of a polymer obtained by polymerizing ethylenically unsaturated monomers wherein at least some of the ethylenically unsaturated monomers are vinyl ester monomers, in combination with at least one lipophilic particulate, one hydrophilic particulate, or mixtures thereof.

37. The composition of claim 36 wherein the lipophilic particulate comprises a pigment, filler, fiber, or mixtures thereof.

38. The composition of claim 37 wherein the hydrophilic particulate comprises a pigment, filler, fiber, or mixtures thereof.

39. The composition of claim 36 wherein at least some of the vinyl ester monomers are fatty esters.

40. The composition of claim 39 wherein the fatty esters are straight or branched chain fatty esters.

41. The composition of claim 39 wherein the fatty esters have at least some unsaturation in the alkyl chain.

42. The composition of claim 39 wherein the polymer comprises at least about twenty percent by weight of the total polymer of vinyl ester monomers.

43. The composition of claim 39 wherein the vinyl ester monomer comprises vinyl fatty alkyl ester monomers wherein the fatty chain has from about 6 to 30 carbon atoms.

44. The composition of claim 44 wherein the fatty ester group in the vinyl ester monomers is selected from the group consisting of hexanoate, heptanoate, octanoate, nonanoate, isononanoate, decanoate, dodecanoate, palmitate, myristate, oleate, caprylate, stearate, isostearate, laurate, behenate, and mixtures thereof.

45. The composition of claim 39 wherein the film forming polymer is a homopolymer of vinyl fatty alkyl ester monomers.

46. The composition of claim 39 wherein the film forming polymer is a copolymer of vinyl fatty alkyl ester monomers and one or more monomers selected from the group consisting of ethylenically unsaturated monomers that are not vinyl esters, silicon monomers, organic groups, and mixtures thereof.

47. The composition of claim 39 wherein the film forming copolymer is a copolymer of vinyl fatty alkyl ester monomers and one or more ethyenically unsaturated monomers that are not vinyl ester monomers.

48. The composition of claim 47 wherein the other ethylenically unsaturated monomers are acrylic acid, methacrylic acid, or their simple esters.

49. The composition of claim 39 wherein the vinyl ester monomers are C₁₋₅ alkyl ester monomers.

50. The composition of claim 39 wherein the vinyl ester monomer comprise vinyl propionate and the polymer is polyvinyl propionate.

51. The composition of claim 39 wherein the monomer comprises vinyl pivilate and the polymer is polyvinyl pivilate.

52. The composition of claim 39 wherein the monomer comprises vinyl neononanoate and the polymer is polyvinylneononanoate.

53. The composition of claim 39 wherein the monomer comprises vinyl neodecanoate and the polymer is polyvinylneodecanoate.

54. The composition of claim 39 wherein the vinyl ester monomer comprises an ethylenically unsaturated monomer having an OCOM group attached to one of the carbon atoms in the ethylenically unsaturated group, wherein O is oxygen, C is carbon, and M is a substituted or unsubstituted straight or branched chain C₁₋₃₀ alkyl wherein the substituents are halogen, hydroxyl, alkylcarbonyloxy, alkyloxycarbonyl,

55. The composition of claim 39 wherein the particulate comprises a hydrophilic particulate.

56. The composition of claim 55 wherein the hydrophilic particulate comprises iron oxides or organic pigments.

57. The composition of claim 39 wherein the particulate comprises a lipophilic particulate.
